(19) United States
(12) Patent Application Publication
(10) Pub. No.: US 2023/0380970 A1
(43) Pub. Date: Nov. 30, 2023

(54) HIGH STRENGTH CORDS FOR CARDIAC PROCEDURES

(71) Applicant: EDWARDS LIFESCIENCES CORPORATION, Irvine, CA (US)

(72) Inventors: Stephen Cournane, Severn, MD (US); Felino V. Cortez, JR., Bowie, MD (US); Luke Anthony Zanetti, Parkton, MD (US)

(21) Appl. No.: 18/447,241

(22) Filed: Aug. 9, 2023

Related U.S. Application Data
(63) Continuation of application No. PCT/US22/15738, filed on Feb. 9, 2022.
(60) Provisional application No. 63/224,627, filed on Jul. 22, 2021.
(60) Provisional application No. 63/147,414, filed on Feb. 9, 2021.

(51) Int. Cl. A61F 2/24

(52) U.S. Cl. CPC A61F 2/2457; A61F 2310/00011; A61F 2310/00179; A61F 2310/00389

(57) ABSTRACT

Described herein are cords (or sutures) and methods for using cords wherein the cords comprise a synthetic aromatic polyamide (or aramid) polymer, wherein the cords comprise a core of a high-strength material such as a polymer (e.g., PET), aramid, ceramic, or metal with a coating of ePTFE encapsulating the core, or wherein the cords comprise braided strands of ePTFE. The disclosed cords have higher strength and durability than typical ePTFE cords or sutures. Furthermore, disclosed herein are methods that utilize the disclosed cords in cardiac repairs, thereby resulting in repairs that are superior to repairs that utilize typical ePTFE cords.

### CROSS-REFERENCE TO RELATED APPLICATIONS

This application is a continuation of International Patent Application No. PCT/US2022/015738, filed Feb. 9, 2022, which claims the benefit of U.S. Patent Application No. 63/224,627, filed Jul. 22, 2021 and of U.S. Patent Application No. 63/147,414, filed Feb. 9, 2021, the entire discloses all of which are incorporated by reference for all purposes.

### BACKGROUND

### Field

Some examples described herein relate to the use of high strength cords for cardiac procedures.

### Description of Related Art

Various disease processes can impair the proper functioning of one or more of the valves of the heart. These disease processes include degenerative processes (e.g., Barlow's disease, fibroelastic deficiency), inflammatory processes (e.g., rheumatic heart disease), and infectious processes (e.g., endocarditis). Additionally, damage to the ventricle from prior heart attacks (e.g., myocardial infarction secondary to coronary artery disease) or other heart diseases (e.g., cardiomyopathy) can distort the geometry of the heart causing valves in the heart to dysfunction. The vast majority of patients undergoing valve surgery, such as mitral valve surgery, suffer from a degenerative disease that causes a malfunction in a leaflet of the valve, which results in prolapse and regurgitation.

Valve regurgitation occurs when the leaflets of the valve do not close completely thereby allowing blood to leak back into the prior chamber when the heart contracts. This may be caused by dilation of the annulus (Carpentier type I malfunction), prolapse of a segment of one or both leaflets above the plane of coaptation (Carpentier type II malfunction), or restriction of the motion of one or more leaflets such that the leaflets are abnormally constrained below the level of the plane of the annulus (Carpentier type II malfunction). Mitral valve regurgitation (MR) results in a volume overload on the left ventricle which in turn progresses to ventricular dilation, decreased ejection performance, pulmonary hypertension, symptomatic congestive heart failure, atrial fibrillation, right ventricular dysfunction and death.

Malfunctioning valves may either be repaired or replaced. Replacement typically involves replacing the patient's malfunctioning valve with a biological or mechanical substitute. Repair typically involves the preservation and correction of the patient's own valve. Successful surgical mitral valve repair restores mitral valve competence, abolishes the volume overload on the left ventricle, improves symptom status, and prevents adverse left ventricular remodeling.

In many instances of mitral valve regurgitation, repair is preferable to valve replacement. Many surgeons have moved to a “non-resectional” repair technique where artificial chordae tendineae (“cords”) made of expanded polytetrafluoroethylene (“ePTFE”) suture, or another suitable material, are placed in the prolapsed leaflet and secured to the heart in the left ventricle, normally to the papillary muscle. Another technique, developed by Dr. Alfieri, involves securing the midpoint of both leaflets together to create a double orifice valve, known as an “edge-to-edge” repair or an Alfieri procedure. Another technique, in addition to or instead of creating the edge-to-edge relationship, includes securing together sutures extending from the leaflets to pull or to otherwise move the posterior annulus towards the anterior leaflet and/or the anterior annulus towards to posterior leaflet to reduce the distance between the anterior annulus and the posterior annulus (or the septal-lateral distance).

### SUMMARY

Described herein are high strength sutures or cords for cardiac procedures. In particular, described herein are materials and combinations of materials for cords implanted as artificial cords (e.g., replacing or supplementing native chordae tendineae) for repairing cardiac valves. In some instances, the cord material is a synthetic aromatic polyamide (or aramid) polymer. In certain implementations, the cord material can be braided or twisted and/or may include core material within the braided or twisted material. In various implementations, the cord material a plurality of ePTFE strands braided together. These strands can have a variety of cross-sectional shapes including, but not limited to, round, square, rectangular, etc. Braiding can be done in conjunction with one or more of the disclosed high-strength cords to enhance endothelialization and/or to improve biostability. In some instances, the cord material includes a core made of a synthetic aramid, a high-strength polymer such as polyethylene terephthalate (PET), a ceramic, and/or a metal with a coating of ePTFE or other suitable material.

In a first aspect, the present disclosure provides a method for repairing a cardiac valve. The method includes attaching a high strength cord to targeted tissue of a heart, the high strength cord including a distal anchor and a suture extending proximally from the distal anchor implant, the high strength cord having a tensile strength of at least 2000 MPa. The method also includes anchoring a proximal end of the high strength cord to the heart.

In some examples of the first aspect, the high strength cord consists of synthetic aramid polymer fibers. In some examples of the first aspect, the high strength cord comprises braided or twisted strands of a synthetic aramid polymer. In further examples, the braided or twisted strands of the synthetic aramid polymer surround a core structure. In further examples, the synthetic aramid strands cover at least 50% of the core structure. In some examples of the first aspect, the high strength cord consists of ePTFE fibers braided or twisted together.

In some examples of the first aspect, the high strength cord comprises a core portion of a high-strength material and a coating material coating the core portion, the coating material configured to improve biostability. In some examples of the first aspect, the coating material fully or partially coats or encapsulates the core portion. In further examples, the high-strength material comprises synthetic aramid polymer fibers. In further examples, the high-strength material comprises a high-strength polymer such as PET. In further examples, the high-strength material comprises a metal. In further examples, the high-strength material comprises a ceramic. In further examples, the coating material comprises ePTFE.

In some examples of the first aspect, the high strength cord comprises a core portion of a high strength material and a jacket portion surrounding the core portion. In further examples, the high strength material comprises a high strength polymer. In further examples, the high strength material comprises a metal. In further examples, the high strength material comprises a ceramic. In some examples of the first aspect, the jacket portion comprises expanded polytetrafluoroethylene. In further examples, the jacket portion is formed from ribbons of flattened expanded polytetrafluoroethylene.

In some examples of the first aspect, anchoring the proximal end includes securing the proximal end to an external wall of the heart. In some examples of the first aspect, anchoring the proximal end includes securing the proximal end to a papillary muscle of the heart. In some examples of the first aspect, the targeted tissue includes a leaflet of a mitral valve. In some examples of the first aspect, the distal anchor is a bulky knot formed using the high strength cord. In some examples of the first aspect, the distal anchor is a barb secured to a distal end of the high strength cord.

In a second aspect, the present disclosure provides a high strength cord for use in cardiac valve repairs. The cord includes synthetic aramid fibers forming a suture and having a tensile strength of at least about 2000 MPa.

In some examples of the second aspect, the synthetic aramid polymer fibers are braided. In further examples, the braided synthetic aramid fibers surround a core material. In further examples, the synthetic aramid strands cover at least 50% of the core structure.

In some examples of the second aspect, the cord further includes a coating of ePTFE material. In further examples, at least about 90% of a cross-section area of the cord comprises the synthetic aramid fibers and less than or equal to about 10% of the cross-section area of the cord comprises the ePTFE material. In some implementations, the cross-sectional ratio of core material to outer layer may vary.

In some examples of the second aspect, a diameter of the cord is less than or equal to about 0.5 mm (about 0.02 inch). In some examples of the second aspect, the cord further includes a coating on the synthetic aramid fibers, the coating configured to improve biostability.

In some examples of the second aspect, the cord further includes a jacket surrounding the synthetic aramid fibers. In further examples, the jacket comprises ePTFE.

In a third aspect, the present disclosure provides a high strength cord for use in cardiac valve repairs. The cord includes a core portion comprising a high-strength material. The cord includes a sheath portion surrounding the core portion, the sheath portion comprising expanded polytetrafluoroethylene material. The cord has a tensile strength of at least about 2000 MPa.

In some examples of the third aspect, the high-strength material of the core portion comprises synthetic aramid fibers. In some examples of the third aspect, the high-strength material of the core portion comprises a metal.

In some examples of the third aspect, the high strength material comprises a high strength polymer. In further examples, the high strength polymer comprises polyethylene terephthalate.

In some examples of the third aspect, the sheath portion is formed from ribbons of flattened ePTFE material. In further examples, the ribbons are wrapped around the core portion and fused.

In some examples of the third aspect, a cross-section area of the core portion is at least about 90% of a cross-section area of the cord and a cross-section area of the sheath portion is less than or equal to about 10% of the cross-section area of the cord. In some examples of the third aspect, a diameter of the cord is less than or equal to about 0.5 mm (about 0.02 inch). In some examples of the third aspect, the sheath portion covers at least 50% of the core portion.

In a fourth aspect, the present disclosure provides a high strength cord for use in cardiac procedures. The cord includes braided ePTFE strands.

In a fifth aspect, the present disclosure provides a high strength cord for use in cardiac procedures. The cord includes a high-strength material as a core. The cord includes a sheath comprising ePTFE wrapped around the core.

In some examples of the fifth aspect, the sheath is fused to the core. In some examples of the fifth aspect, the core includes a high-strength polymer. In further examples, the high-strength polymer comprises a polyester (e.g., PET). In some examples of the fifth aspect, the thickness of the sheath is less than or equal to about 0.1 mm (about 0.005 inch). In some examples of the fifth aspect, the diameter of the core material is less than or equal to 0.2 mm (about 0.01 inch).

It should be understood that any methods disclosed herein, including methods for treating a human or non-human patient, also encompass analogous methods for training, device development, method development, teaching and the like that can be performed on an anthropogenic ghost or other simulated patient. Suitable simulated patients can include any combination of physical and virtual components. Any component can be independently animated or static. Examples of suitable physical components include cadavers, human or non-human; portions of cadavers, including organ systems, isolated organs, or tissue; and synthetic or man-made components. Virtual components can include visual content, for example images provided on a screen, projected on an object, holograms, and the like; as well other sensory simulations, including auditory, tactile, and olfactory stimuli.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

FIG. 1 illustrates a cut-away anterior view of a heart, showing the internal chambers, valves and adjacent structures.

FIG. 2A illustrates a top perspective view of a healthy mitral valve with the mitral leaflets closed.

FIG. 2B illustrates a top perspective view of a dysfunctional mitral valve with a visible gap between the mitral leaflets.

FIG. 2C illustrates a cross-sectional view of a heart illustrating a mitral valve prolapsed into the left atrium.

FIG. 2D illustrates an enlarged view of the prolapsed mitral valve of FIG. 2C.

FIG. 3 illustrates a cross-sectional view of a heart showing the left atrium, right atrium, left ventricle, right ventricle and the apex region.

FIG. 4 illustrates a valve repair using a high strength cord.

FIG. 5 is a schematic illustration of using high strength cords with bulky knots as anchors to repair a mitral valve with leaflets that are separated by a gap.

FIG. 6 illustrates an example of an annuloplasty ring that has a core surrounded by a jacket, the core comprising any of the high-strength cords disclosed herein.

FIG. 7 illustrates an example sub-valvular procedure using any of the high strength cords disclosed herein.

FIG. 8 illustrates using a high strength cord in a procedure to reshape a portion of the heart.

FIG. 9 illustrates using a high strength cord in an annuloplasty procedure that implants the cord in the coronary sinus to reshape the annulus.

FIG. 10 illustrates using a high strength cord in an annuloplasty procedure that implants a plurality of anchors in an annulus and pulls the anchors together using the high strength cord to reshape the annulus.

FIG. 11 illustrates using a high strength cord in an annuloplasty procedure that implants a band in the ventricle under the annulus, the high strength cord configured to cinch the band to reshape the annulus.

FIG. 12 illustrates using a high strength cord in an annuloplasty procedure that implants a band in the atrium at the annulus, the high strength cord configured to cinch the band to reshape the annulus.

FIGS. 13A, 13B, and 13C illustrate examples of high strength cords using synthetic aramid polymer fibers.

FIG. 14 illustrates an example of a high strength cord using a core of a high strength material and an external sheath of ePTFE material.

FIG. 15 illustrates an example of a high strength cord using braided ePTFE strands.

FIG. 16 illustrates an example of a high strength cord using a core of a high strength material and an external sheath of ePTFE material, the sheath being wrapped around the core.

FIG. 17 illustrates a flowchart of an example method for repairing a cardiac valve using any of the high-strength cords disclosed herein.

### DETAILED DESCRIPTION OF SOME EXAMPLES

The headings provided herein, if any, are for convenience only and do not necessarily affect the scope or meaning of the disclosed methods, systems, or devices.

### Overview

Doctors can perform a wide range of surgical procedures on a defective heart valve. In degenerative mitral valve repair procedures, techniques include, for example and without limitation, various forms of re-sectional repair, chordal implantation, ventricular reshaping, and edge-to-edge repairs. Clefts or perforations in a leaflet can be closed and occasionally the commissures of the valve sutured to minimize or eliminate MR. In these and similar procedures, ePTFE cords are typically used for performing the repairs, e.g., as artificial cords and/or sutures. For example, the mitral valve can be repaired by inserting ePTFE cords into the mitral valve and anchoring the cords to the ventricle or papillary muscle. However, it has been reported that ePTFE cords have broken following both open heart repair and less invasive procedures. Accordingly, there is a need for cords with increased strength and durability.

To address these and other issues, disclosed herein are cords (or sutures) and methods for using cords wherein the cords comprise a synthetic aromatic polyamide (or aramid) polymer, wherein the cords comprise a core of a high-strength polymer, composite material, aramid, ceramic, or metal with a coating of ePTFE encapsulating the core, or where the cords comprise braided ePTFE sutures, fibers, or strands. In some examples, the cord is a braided ePTFE suture that comprises multiple strands of ePTFE sutures interlaced in a variety of patterns. In some examples, the cords comprising a synthetic aramid polymer can be coated to improve biostability. The disclosed cords have higher strength and durability than typical ePTFE cords or sutures. Furthermore, disclosed herein are methods that utilize the disclosed cords in valve repairs including implanting artificial cords, sub-valvular techniques, reshaping organs, annuloplasty, and the like. Utilization of the disclosed cords can result in repairs that are superior to repairs that utilize typical ePTFE cords.

The use of the disclosed cords in the vascular system may provide a number of advantages. The disclosed cords and associated methods may be advantageous due at least in part to their increased strength and durability resulting in a lower risk of breakage in a patient and enabling larger annular reductions. These larger annular reductions are due at least in part to the increased strength of the cords, allowing for greater forces to be applied to the annulus and/or leaflets. Advantages also include, and may be particularly pronounced for examples that utilize ePTFE material, proven long-term reliability durability, ability to retain strength after deformation, elasticity and flexibility, softness and pliability, nonabrasive to tissues, biostable/biocompatible, microporous and easily endothelializable to establish long term biocompatibility, material does not trigger concomitant inflammation or immune reaction, material does not promote thrombus formation, and easy to handle and tie.

These features are particularly beneficial when used in the vascular system and may not be applicable to the use of synthetic aramid polymer sutures in orthopedic applications (e.g., in repairing ligaments such as the anterior cruciate ligament or ACL). For example, the cords used in the vascular system disclosed herein can have a diameter that is less than or equal to about 0.5 mm (about 0.02″) whereas cords used for orthopedic purposes typically have a larger diameter to increase the strength of the sutures. Moreover, the cords disclosed herein are configured for use in the vascular system, or within the blood stream, making biostability and biocompatibility more important than cords used in orthopedic applications. Consequently, some of the synthetic aramid polymer cords disclosed herein are coated with a material to improve biostability and/or biocompatibility.

In some examples, in addition to valve repairs, the disclosed cords may be advantageous in annuloplasty procedures, sub-valvular procedures, and procedures that are used to re-shape or modify the chambers of the heart and/or other internal organs. For example, the disclosed sutures may be used to pull the ventricle inwards to reduce the volume of the ventricle. The disclosed high strength cords can be used in open heart procedures, less-invasive procedures, minimally invasive procedures, non-invasive procedures, transcatheter approaches, etc. Although principally described in the context of mitral valve repairs, it is to be understood that the disclosed cords can be used in tricuspid valve repairs and other valve repairs.

In some instances, disclosed methods for repairing tissue include inserting a delivery device, such as a delivery device described in the '761 PCT Application and/or in International Patent Application No. PCT/US2016/055170 (published as WO 2017/059426A1 and referred to herein as “the '170 PCT Application”), the entire disclosure of each of which is incorporated herein by reference, into a body and extending a distal end of the delivery device to a proximal side of the tissue. Advancement of the delivery device may be performed in conjunction with sonography or direct visualization (e.g., direct transblood visualization), and/or any other suitable remote visualization technique. Furthermore, one or more steps of the disclosed methods may also be performed in conjunction with any suitable remote visualization technique. With respect to the disclosed methods, one or more parts of a procedure may be monitored in conjunction with transesophageal (TEE) guidance or intracardiac echocardiography (ICE) guidance. For example, this may facilitate and direct the movement and proper positioning of the delivery device for contacting the appropriate target cardiac region and/or target cardiac tissue (e.g., a valve leaflet, a valve annulus, or any other suitable cardiac tissue). Typical procedures for use of echo guidance are set forth in Suematsu, Y., J. Thorac. Cardiovasc. Surg. 2005; 130:1348-56 (“Suematsu”), the entire disclosure of which is incorporated herein by reference.

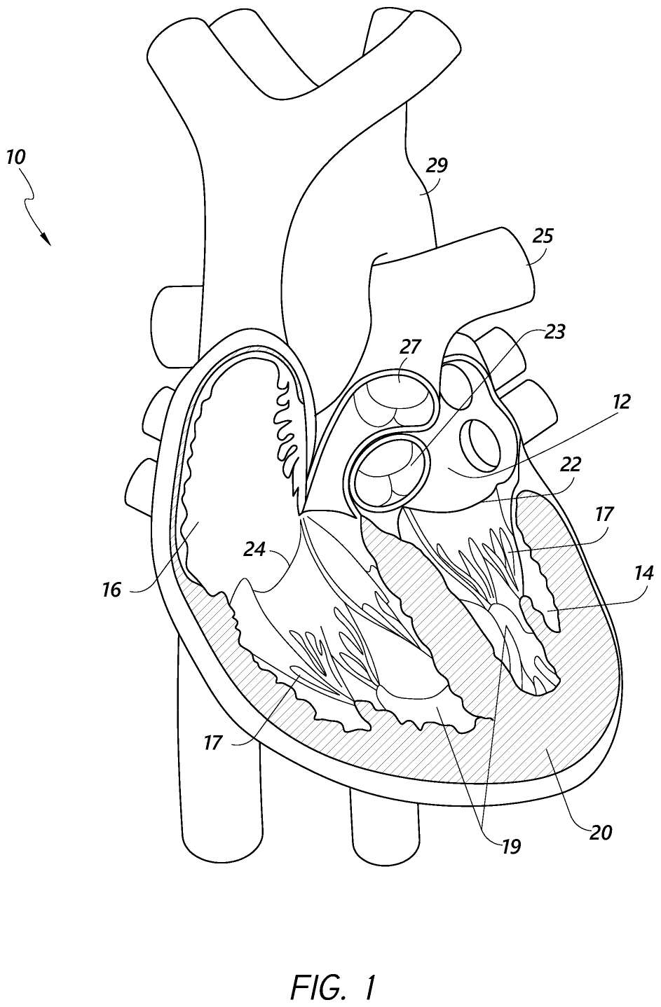

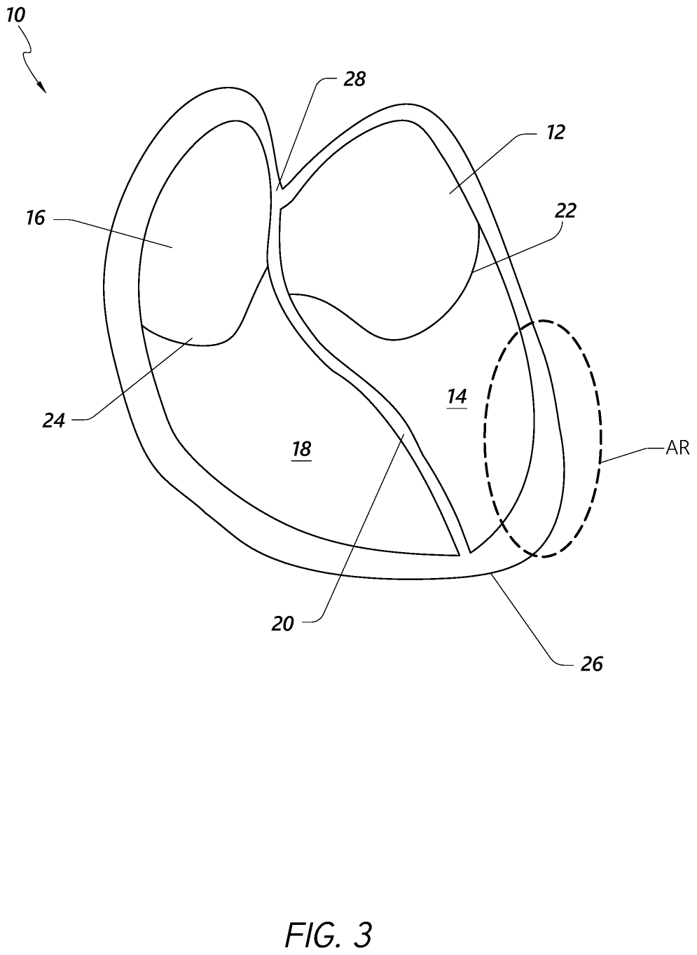

As illustrated in FIG. 1, the human heart 10 has four chambers, which include two upper chambers denoted as atria 12, 16 and two lower chambers denoted as ventricles 14, 18. A septum 20 (see, e.g., FIG. 3) divides the heart 10 and separates the left atrium 12 and left ventricle 14 from the right atrium 16 and right ventricle 18. The heart further contains four valves 22, 23, 26, and 27. The valves function to maintain the pressure and unidirectional flow of blood through the body and to prevent blood from leaking back into a chamber from which it has been pumped.

Two valves separate the atria 12, 16 from the ventricles 14, 18, denoted as atrioventricular valves. The mitral valve 22, also known as the left atrioventricular valve, controls the passage of oxygenated blood from the left atrium 12 to the left ventricle 14. A second valve, the aortic valve 23, separates the left ventricle 14 from the aortic artery (aorta) 29, which delivers oxygenated blood via the circulation to the entire body. The aortic valve 23 and mitral valve 22 are part of the “left” heart, which controls the flow of oxygen-rich blood from the lungs to the body. The right atrioventricular valve, the tricuspid valve 24, controls passage of deoxygenated blood into the right ventricle 18. A fourth valve, the pulmonary valve 27, separates the right ventricle 18 from the pulmonary artery 25. The right ventricle 18 pumps deoxygenated blood through the pulmonary artery 25 to the lungs wherein the blood is oxygenated and then delivered to the left atrium 12 via the pulmonary vein. Accordingly, the tricuspid valve 24 and pulmonic valve 27 are part of the right heart, which control the flow of oxygen-depleted blood from the body to the lungs.

Both the left and right ventricles 14, 18 constitute pumping chambers. The aortic valve 23 and pulmonic valve 27 lie between a pumping chamber (ventricle) and a major artery and control the flow of blood out of the ventricles and into the circulation. The aortic valve 23 and pulmonic valve 27 have three cusps, or leaflets, that open and close and thereby function to prevent blood from leaking back into the ventricles after being ejected into the lungs or aorta 29 for circulation.

Both the left and right atria 12, 16 are receiving chambers. The mitral valve 22 and tricuspid valve 24, therefore, lie between a receiving chamber (atrium) and a ventricle to control the flow of blood from the atria to the ventricles and prevent blood from leaking back into the atrium during ejection from the ventricle. Both the mitral valve 22 and tricuspid valve 24 include two or more cusps, or leaflets (not shown in FIG. 1), that are encircled by a variably dense fibrous ring of tissues known as the annulus (not shown in FIG. 1). The valves are anchored to the walls of the ventricles by chordae tendineae (chordae) 17. The chordae tendineae 17 are cord-like tendons that connect the papillary muscles 19 to the leaflets (not shown in FIG. 1) of the mitral valve 22 and tricuspid valve 24 of the heart 10. The papillary muscles 19 are located at the base of the chordae tendineae 17 and are within the walls of the ventricles. The papillary muscles 19 do not open or close the valves of the heart, which close passively in response to pressure gradients; rather, the papillary muscles 19 brace the valves against the high pressure needed to circulate the blood throughout the body. Together, the papillary muscles 19 and the chordae tendineae 17 are known as the sub-valvular apparatus. The function of the sub-valvular apparatus is to keep the valves from prolapsing into the atria when they close.

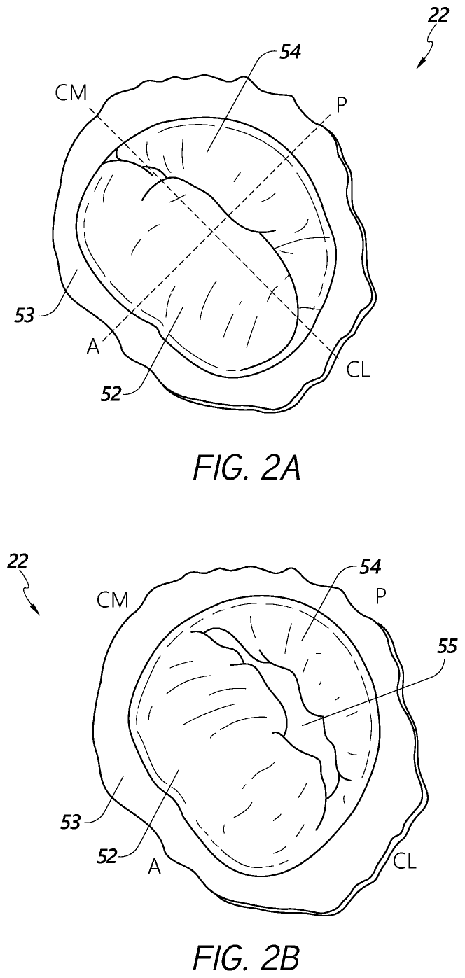

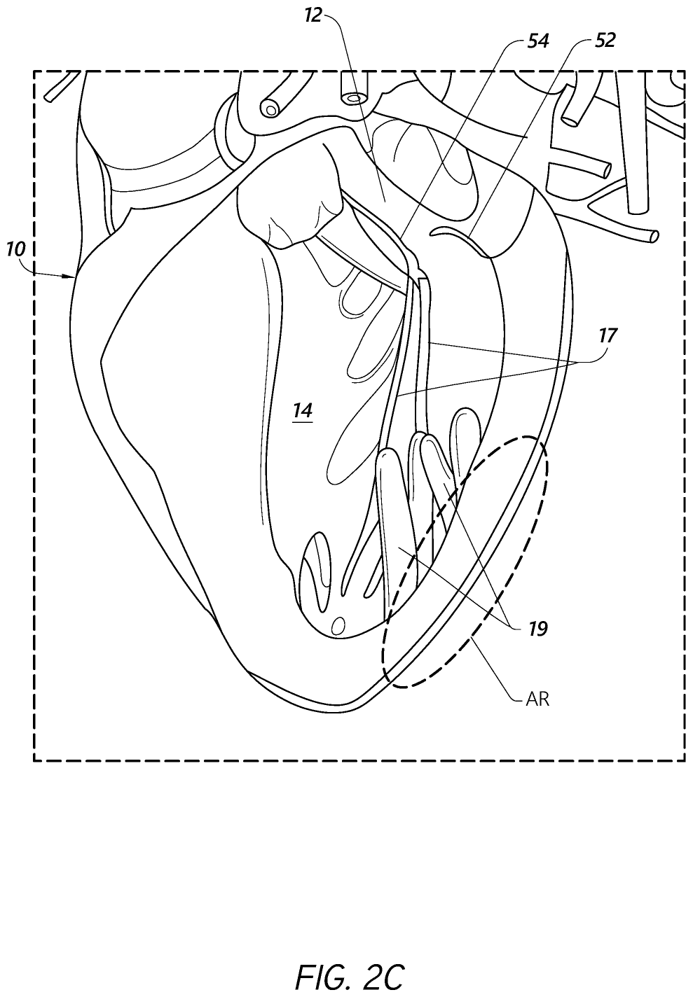

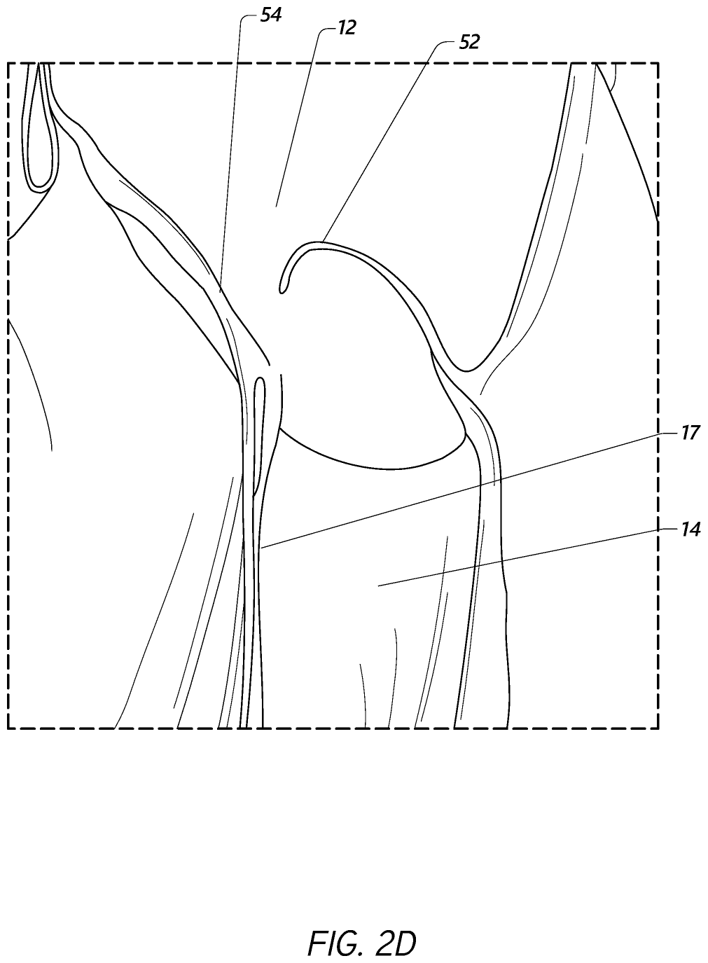

The mitral valve 22 is illustrated in FIG. 2A. The mitral valve 22 includes two leaflets, the anterior leaflet 52 and the posterior leaflet 54, and a diaphanous incomplete ring around the valve, called the annulus 53. The mitral valve 22 has two papillary muscles 19, the anteromedial and the posterolateral papillary muscles (see, e.g., FIG. 1), which attach the leaflets 52, 54 to the walls of the left ventricle 14 via the chordae tendineae 17 (see, e.g., FIG. 1).

FIG. 2B illustrates a prolapsed mitral valve 22. As can be seen with reference to FIGS. 2B-2D, prolapse occurs when a prolapsed segment of a leaflet 52, 54 of the mitral valve 22 is displaced above the plane of the mitral annulus into the left atrium 12 (see FIGS. 2C and 2D) preventing the leaflets from properly sealing together to form the natural plane or line of coaptation between the valve leaflets during systole. Because one or more of the leaflets 52, 54 malfunctions, the mitral valve 22 does not close properly, and, therefore, the leaflets 52, 54 fail to coapt. This failure to coapt causes a gap 55 between the leaflets 52, 54 that allows blood to flow back into the left atrium, during systole, while it is being ejected by the left ventricle. As set forth above, there are several different ways a leaflet may malfunction, which can thereby lead to regurgitation.

Mitral valve regurgitation increases the workload on the heart and may lead to very serious conditions if left untreated, such as decreased ventricular function, pulmonary hypertension, congestive heart failure, permanent heart damage, cardiac arrest, and ultimately death. Since the left heart is primarily responsible for circulating the flow of blood throughout the body, malfunction of the mitral valve 22 is particularly problematic and often life threatening.

Disclosed herein are high strength cords suitable for use in procedures to repair a cardiac valve, such as a mitral valve. Such procedures include procedures to repair regurgitation that occurs when the leaflets of the mitral valve do not coapt at peak contraction pressures, resulting in an undesired back flow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed to effectuate a cardiac valve repair, which will depend on the specific abnormality and the tissues involved. The procedures can include open heart procedures, less-invasive procedures, minimally invasive procedures, non-invasive procedures, procedures employing a transcatheter approach, etc. The procedures can include, for example and without limitation, implantation of artificial cords, annuloplasty, sub-valvular techniques (e.g., manipulating elements of the sub-valvular apparatus), reshaping of the heart, and the like.

FIG. 3 illustrates that one or more chambers, e.g., the left atrium 12, left ventricle 14, right atrium 16, or right ventricle 18, in the heart 10 may be accessed in accordance with any suitable method including open heart procedures, less-invasive procedures, minimally invasive procedures, non-invasive procedures, transcatheter approaches, etc. Access into a chamber 12, 14, 16, 18 in the heart 10 may be made at any suitable site of entry. In some examples, less-invasive procedures and non-invasive procedures can preferably gain access to the desired chamber of the heart through the apex region of the heart, for example, slightly above the apex 26 at the level of the papillary muscles 19 (see also FIG. 2C). Typically, access into the left ventricle 14 (e.g., to perform a mitral valve repair) is gained through the apical region, close to (or slightly skewed toward the left of) the median axis 28 of the heart 10. Typically, access into the right ventricle 18 (e.g., to perform a tricuspid valve repair) is gained through the apical region, close to or slightly skewed toward the right of the median axis 28 of the heart 10. Generally, an apex region of the heart is a bottom region of the heart that is within the left or right ventricular region and is below the mitral valve 22 and tricuspid valve 24 and toward the tip or apex 26 of the heart 10. More specifically, an apex region AR of the heart (see, e.g., FIG. 3) is within a few centimeters to the right or to the left of the septum 20 of the heart 10 at or near the level of the papillary muscles 19. Accordingly, the ventricle can be accessed directly via the apex 26, or via an off-apex location that is in the apical or apex region AR, but slightly removed from the apex 26, such as via a lateral ventricular wall, a region between the apex 26 and the base of a papillary muscle 19, or even directly at the base of a papillary muscle 19 or above.

The mitral valve 22 and tricuspid valve 24 can be divided into three parts: an annulus (see 53 in FIGS. 2A and 2B), leaflets (see 52, 54 in FIGS. 2A and 2B), and a sub-valvular apparatus. The sub-valvular apparatus includes the papillary muscles 19 (see FIG. 1) and the chordae tendineae 17 (see FIG. 1), which can elongate and/or rupture. If the valve is functioning properly, when closed, the free margins or edges of the leaflets come together and form a tight junction, the are of which, in the mitral valve, is known as the line, plane or area of coaptation. Normal mitral and tricuspid valves open when the ventricles relax allowing blood from the atrium to fill the decompressed ventricle. When the ventricle contracts, chordae tendineae properly position the valve leaflets such that the increase in pressure within the ventricle causes the valve to close, thereby preventing blood from leaking into the atrium and assuring that all of the blood leaving the ventricle is ejected through the aortic valve (not shown) and pulmonic valve (not shown) into the arteries of the body. Accordingly, proper function of the valves depends on a complex interplay between the annulus, leaflets, and sub-valvular apparatus. Lesions in any of these components can cause the valve to dysfunction and thereby lead to valve regurgitation. As set forth herein, regurgitation occurs when the leaflets do not coapt properly at peak contraction pressures. As a result, an undesired back flow of blood from the ventricle into the atrium occurs.

Although the procedures described herein are with reference to repairing a cardiac mitral valve or tricuspid valve using artificial cords, the cords and methods presented are readily adaptable for various types of tissue, leaflet, and annular repair procedures. In general, the methods herein are described with reference to a mitral valve 22 but should not be understood to be limited to procedures involving the mitral valve.

Examples of Cardiac Repairs using High Strength Cords

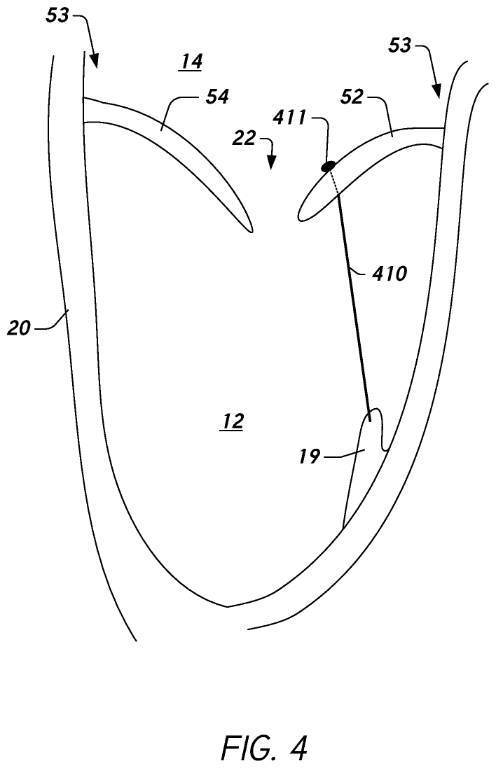

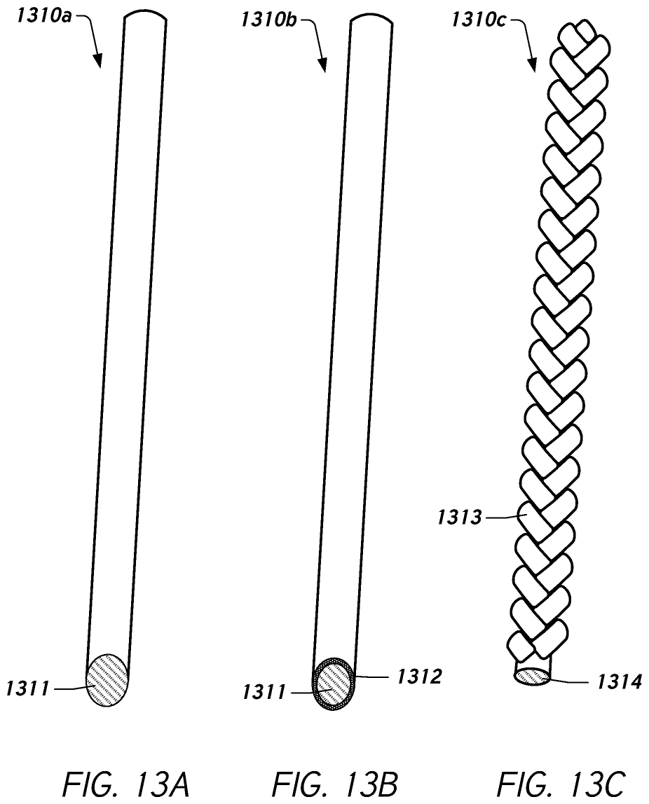

FIG. 4 illustrates the use of one or more artificial cords 410 to repair a mitral valve 22. Although described with respect to repairing the mitral valve, the disclosed cords can be used to repair a tricuspid valve or other cardiac valve. The one or more cords 410 are high strength cords, examples of which are disclosed in greater detail herein with respect to FIGS. 13-16. The mitral valve 22 can be repaired by inserting the high strength cords 410 into the mitral valve 22 and anchoring the cords 410 to the ventricle 12 and/or papillary muscle 19. In some examples, the cords 410 can be anchored to the septum 20. The cords 410 can be attached to the anterior leaflet 52 using a distal anchor 411. In some examples, the cords 410 can be attached to the posterior leaflet 54 and/or the annulus 53. The distal anchor 411 can be any suitable anchor including, for example, hooks, barbs, knots, grafts, fabric, etc., or any combination thereof. The distal anchor 411 can be formed of any suitable material. In some instances, for example, the material of the distal anchor 411 can be the same as the cords 410 or can be any one or more of ePTFE sutures, polybutylate-coated polyester sutures, or polyester sutures (such as, for example, ETHIBOND EXCEL® polyester sutures).

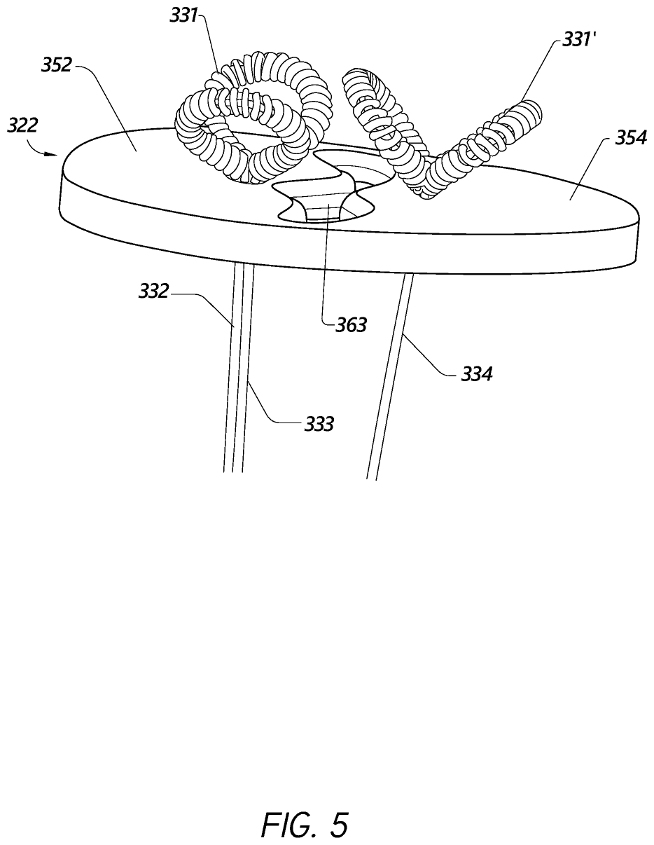

In some examples, the distal anchor 411 can be made from a distal portion of the cords 410, e.g., a bulky knot, an example of which is described herein with respect to FIG. 5. In some examples, the distal portion of the cords 410 can be secured to the annulus 53 and/or the posterior leaflet 54 in addition to or as an alternative to the anterior leaflet 52.

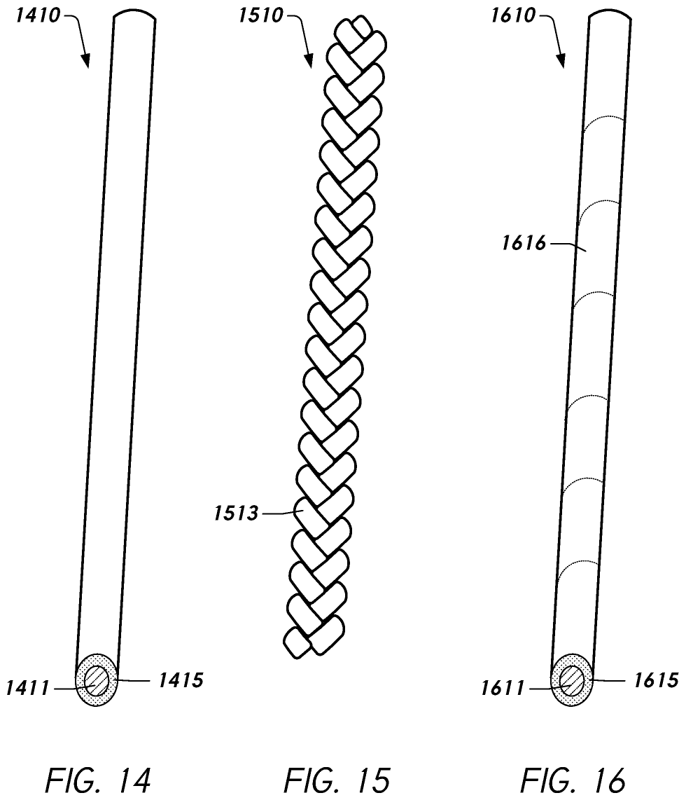

The cords 410 can be any of the high strength cords described herein. For example, the cords 410 can comprise synthetic aramid fibers, as described herein with reference to FIGS. 13A-13C. In such examples, the synthetic aramid fibers can be braided or twisted and/or other structures can be interwoven with the aramid fibers, as described herein. As another example the cords 410 can comprise a core of a high-strength polymer, composite material, ceramic, or metal surrounded by a sheath, coating, or jacket of ePTFE material, as described herein with reference to FIGS. 14 and 16. As another example, the cords 410 can comprise braided ePTFE strands, as described herein with reference to FIG. 15.

The materials used in the high-strength cords 410 are configured to be strong and to have relatively high fatigue resistance. When referring to high strength cords herein, the term high strength can refer to high tensile strength, high modulus, and/or high tenacity. The strength of the cords 410 is high relative to sutures made primarily or exclusively using polyester (e.g., ETHIBOND EXCEL® polyester suture, Johnson & Johnson) or ePTFE material (e.g., GORE-TEX® ePTFE suture, W.L. Gore). Byway of example, GORE-TEX® sutures can have knot-pull tensile strengths ranging from about 3 N (CV-8 size suture) to about 53 N (CV-o size suture). In contrast, a suitable aramid fiber KEVLAR® 119 aramid has a tensile strength of about 350 N (straight test on yarn) and 360 N (loop test on yarn). Different types of KEVLAR® aramid have tensile strengths that range from about 300 N to about 370 N (straight test) and from about 230 N to about 360 N (loop test). Put another way, the tensile strength of ePTFE sutures (accounting for differing diameters of the sutures) is typically between about 30 MPa and about 60 MPa whereas the tensile strength of synthetic aramid polymers (e.g., KEVLAR® aramid, DuPont) is at least about 2000 MPa and can exceed 3600 MPa. Thus, the high-strength cords 410 can be characterized as having a tensile strength that is higher than ePTFE sutures by a factor of at least about 3, a factor of at least about 5, at least about 7, or at least about 10. The high-strength cords 410 may also be characterized as having a tensile strength that is at least about 100 N, at least about 200 N, at least about 300 N, or at least about 350 N. The high-strength cords 410 may also be characterized as having a tensile strength that is at least about 2000 MPa, at least about 3000 MPa, at least about 3500 MPa, or at least about 3600 MPa. The disclosed sutures may also be more thermally stable than typical ePTFE sutures. For example, synthetic aramid polymers do not see a significant degradation in strength in the temperature range from about 20° C. to about 37° C. whereas ePTFE reduces strength by about 25% over this same temperature range.

Advantageously, using a stronger material for the cords 410, such as a synthetic aromatic polyamide polymer or a high-strength material surrounded by ePTFE material or braided ePTFE strands, improves the durability of the artificial cords. Furthermore, increased strength and durability enables greater forces to be applied in cardiac valve repairs. This allows, for example, greater deformation of the annulus 53 (e.g., larger annular reductions) and/or larger forces on the leaflets 52, 54 with little or no increase in the risk of cord breakage.

FIG. 5 is a schematic illustration of a mitral valve 322 with leaflets 352, 354 that are separated by a gap 363. Two bulky knots implants or anchors 331, 331′ are disposed on an atrial, distal, or top side of the leaflets 352, 354, respectively. Sutures 332, 333, 334 extend proximally from the bulky knots 331, 331′. The sutures 332, 333, 334 are high-strength sutures, as described herein. For example, the sutures 332, 333, 334 can comprise synthetic aramid fibers (e.g., KEVLAR® aramid, Dupont; TWARON® aramid, Teijin; NOMEX® meta-aramid, Dupont), as described in greater detail herein with respect to FIGS. 13A-13C, or can comprise a high-strength core (e.g., high-strength polymer, composite material, synthetic aramid polymer fibers, ceramic, and/or metal) surrounded by ePTFE material, as described in greater detail herein with respect to FIGS. 14 and 16, or can comprise braided ePTFE strands, as described in greater detail herein with respect to FIG. 15.

The anchors 331, 331′ can be formed with the same suture material as the sutures 332, 333, 334. The suture material for the anchors 331, 331′ forms one or more loops on the atrial side of the leaflets 352, 354 and extends through the leaflets 352, 354, with two loose suture end portions 332, 333, 334 that extend on the ventricular, proximal, or bottom side of the leaflets 352, 354. The implant 331 has suture end portions 332 and 333, and the implant 331′ has suture end portion 334 (and another end portion not shown in FIG. 5). The suture material forming the anchors 331, 331′ can be braided, twisted, or knotted (e.g., with overhand knots) to form the anchors 331, 331′.

After the implants 331, 331′ are in a desired or targeted position (which can be confirmed with imaging, for example), a device can be used to secure the implants 331, 331′ in the desired position and to secure the valve leaflets 352, 354 in an edge-to-edge relationship. Further, in addition to or instead of creating the edge-to-edge relationship, to promote a larger surface of coaptation, the implants 331, 331′ can be secured together to pull or otherwise move the posterior annulus towards the anterior leaflet and/or the anterior annulus towards the posterior leaflet, to reduce the distance between the anterior annulus and the posterior annulus, e.g., the septal-lateral distance by about 10%-40%. Approximating the anterior annulus 352 and the posterior annulus 354 in this manner can decrease the valve orifice, and thereby decrease, limit, or otherwise prevent undesirable regurgitation.

Examples of forming distal anchors, pre-formed knots, and/or locking sutures are presented in U.S. Pat. No. 8,852,213, International Patent Publication No. 2017/059426, and U.S. Patent Publication No. 2019/0117401, each of which is incorporated by reference herein in its entirety for all purposes. For each of these anchors and/or knots, the material(s) used can be the same as the material(s) used for the sutures 332, 333, 334, e.g., synthetic aramid polymer fibers or a combination of a high-strength material with an ePTFE coating.

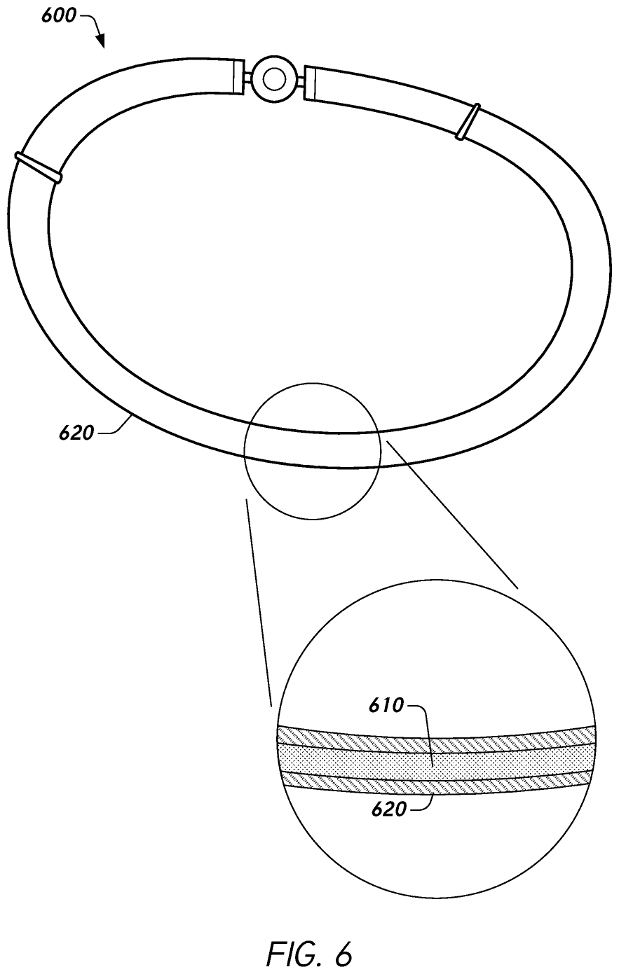

FIG. 6 illustrates an example of an annuloplasty ring 600 that has a core 610 surrounded by a jacket 620, the core 610 comprising any of the high-strength cords disclosed herein. The annuloplasty ring 600 can be used to reshape, reinforce, or tighten the annulus around a heart valve. The annuloplasty ring 600 can be used in a procedure by itself or it can be used in conjunction with other procedures, such as the procedures described herein, to repair a cardiac valve.

The jacket 620 can be made of any suitable combination of durable plastic, metal, and fabric. Suitable materials include, for example and without limitation, silicone rubber, polyester knit fabric, plastic strips, titanium alloys, siloxane polymer rubber, non-magnetic cobalt-chromium-nickel-molybdenum alloy, etc. The jacket 620 can be configured so that the ring 600 has variable flexibility. For example, variable flexibility of the ring 600 allows for physiologic contractility of the valve in which it is implanted during systole. The core 610 can include any of the high strength cords disclosed herein. The core 610 can be a cord of synthetic aramid polymer fibers (coated or uncoated, braided or unbraided) or a high-strength material (e.g., polymer, composite material, ceramic, or metal) encapsulated by ePTFE or braided ePTFE strands. In some examples, the core 610 can be used to adjust the size, shape, tension, etc. of the ring 600 to improve performance of the valve after implantation of the ring 600.

FIG. 7 illustrates an example sub-valvular procedure using one or more high strength cords 710. The cords 710 can be any of the cords described herein with reference to FIGS. 13-16. Sub-valvular procedures include procedures to reposition one or more papillary muscles 19 or manipulation or alteration of one or more chordae tendineae 17 to improve valve function (e.g., to reduce regurgitation).

The illustrated sub-valvular procedure anchors the one or more high strength cords 710 to the papillary muscle 19 and to a wall of the heart to relocate the papillary muscle 19 to improve performance of the chordae tendineae 17. The one or more cords 710 can be anchored to additional papillary muscles 19 of the anterior leaflet 52 and/or to papillary muscles of the posterior leaflet 54. In some examples, a cord 710 can be used to form a loop around multiple papillary muscles to approximate the papillary muscles. In certain examples, a cord 710 can be anchored to another part of the anatomy such as the atrium 14, the annulus 53, or outside the heart.

The illustrated procedure may be generally referred to as papillary muscle relocation. Other similar sub-valvular procedures may also utilize the one or more high strength cords 710. Example sub-valvular procedures that may use the disclosed cords 710 include, for example and without limitation, papillary muscle relocation, papillary muscle sling, papillary muscle approximation, papillary muscle sandwich plasty, “ring and string” procedures, or ring noose string. In some examples, these procedures are performed in conjunction with implantation of an annuloplasty ring, as disclosed herein.

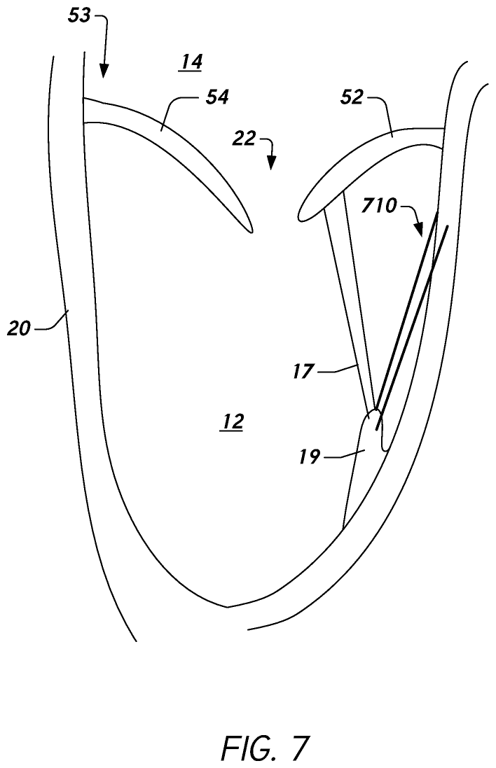

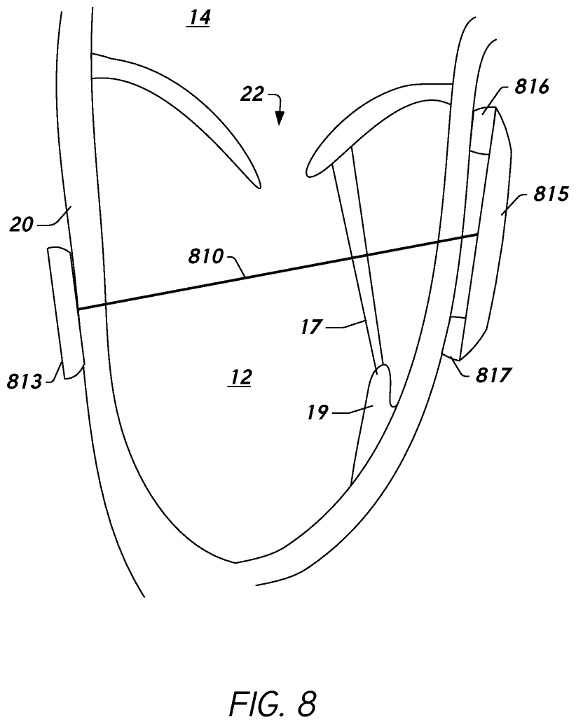

FIG. 8 illustrates using a high strength cord 810 in a procedure to reshape a portion of the heart. The cord 810 can be any of the cords described herein with reference to FIGS. 13-16. To reshape the heart, an anterior pad 813 can be implanted or anchored to an epicardial surface of the heart on one side of a targeted chamber of the heart (e.g., the ventricle 12). A posterior pad 815 can be implanted or anchored to the heart on another side of the targeted chamber. The cord 810 extends between the anterior pad 813 and the posterior pad 815. The length of the cord 810 can be adjusted to approximate the anterior pad 813 and the posterior pad 815 to reshape the targeted chamber. In some examples, the anterior pad 813 is adjustable and is fixed to the cord 810 after sizing the device. In some examples, the posterior pad 815 has a superior head 816 and an inferior head 817 configured to change a shape at the level of the annulus and the level of the papillary muscle, respectively.

In some examples, the transventricular chordal length can be reduced by about 25%. This procedure advantageously can affect and stabilize both the mitral annulus and papillary muscles, can be implanted in an off-pump procedure, can be easily reversed, and has little or no effect on annular dynamics.

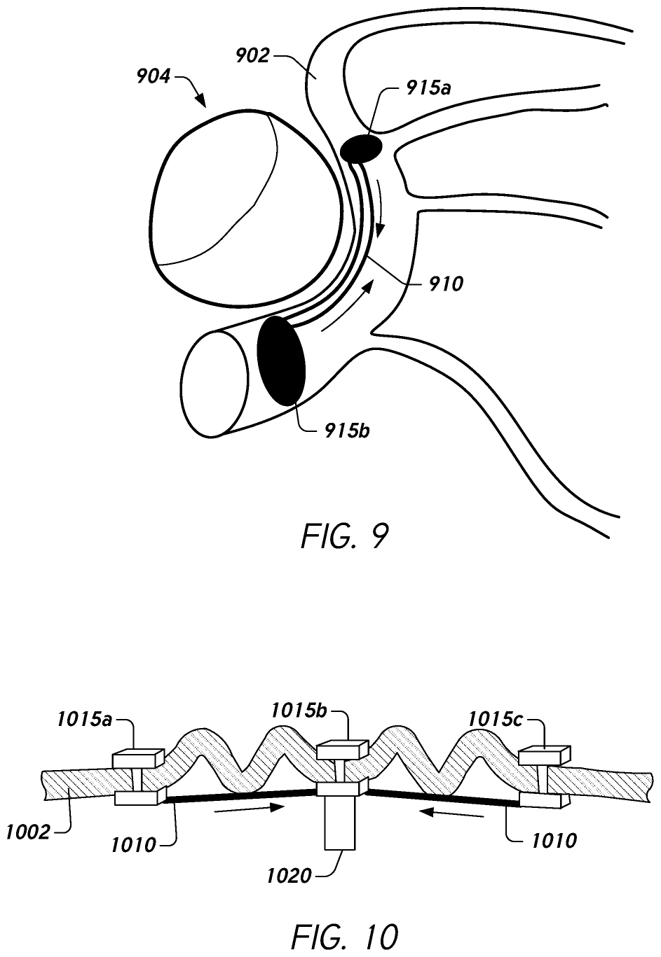

FIG. 9 illustrates using a high strength cord 910 in an annuloplasty procedure that implants the cord 910 in the coronary sinus 902 to reshape the annulus 904 of the mitral valve. The procedure involves implanting a pair of anchors 915a, 915b in the coronary sinus 902, the pair of anchors connected by a cord 910, and then cinching or tightening the cord 910 to reduce the distance between the anchors 915a, 915b, thereby reducing the size and/or altering the shape of the annulus 904 of the mitral valve. This procedure thus uses an intravascular support that is designed to change the shape of the annulus that is adjacent to the coronary sinus in which the support is placed. The support is designed to aid the closure of a mitral valve. The support is placed in the coronary sinus and vessel that are located adjacent the mitral valve and urges the vessel wall against the valve to aid its closure.

FIG. 10 illustrates using a high strength cord 1010 in an annuloplasty procedure that implants a plurality of anchors 1015a-1015c in an annulus 1002 of a mitral valve and pulls the anchors 1015a-1015c together using the high strength cord 1010 to reshape the annulus 1002. In some examples, pledgeted anchors 1015a-1015c are deployed on the posterior mitral annulus at P1P2 and P2P3 locations. These anchors 1015a-1015c are cinched to reduce the annulus 1002 using the high strength cord 1010. The procedure includes anchoring tissue with anchor assemblies 1015a-1015c comprising a proximal end portion, a distal end portion and a compressible intermediate portion located between the proximal and distal end portions and movable between an elongated configuration and a shortened configuration. The procedure includes inserting at least one of the anchor elements 1015a-1015c through the tissue 1002 and pulling the high strength cord 1010 relative to the other anchor elements. This draws the proximal and distal end portions of the anchor assembly toward each other and compresses the intermediate portion into the shortened configuration with the assembly engaged against the tissue. The tissue may comprise the mitral valve annulus and the anchor assemblies may be engaged on opposite sides of the tissue, such as on opposite sides of the mitral valve annulus. The procedure includes drawing the anchor assemblies toward each other to plicate the tissue 1002 whereupon the anchor assemblies are locked relative to each other to lock the plicated condition of the tissue 1002. This procedure may, for example, be repeated any number of times to plicate the posterior portion of the mitral valve annulus for purposes of achieving annuloplasty. A mechanism 1020 can be used to cinch the high strength cord to draw the anchor assemblies toward each other.

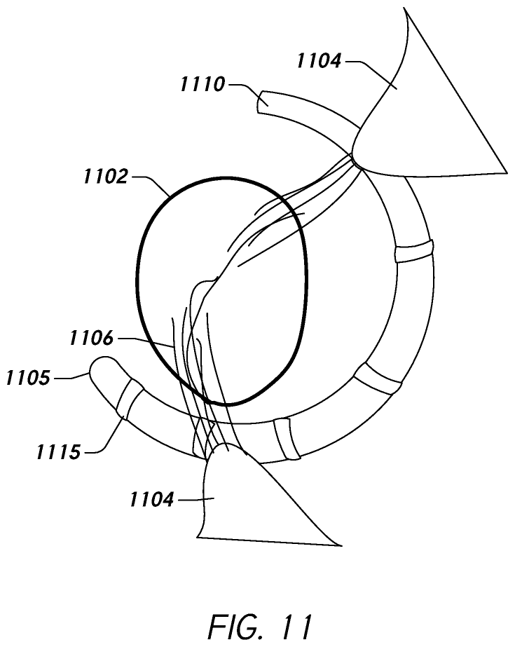

FIG. 11 illustrates using a high strength cord 1110 in an annuloplasty procedure that implants a band 1105 in the ventricle under the annulus 1102, the high strength cord 1110 configured to cinch the band 1105 to reshape the annulus 1102. The band 1105 is implanted above the papillary muscles 1104 and out of the way of the natural chords 1106. The band 1105 includes a plurality of anchors 1115 to anchor the band 1105 in the ventricle.

The procedure generally involves contacting an anchor delivery device with a length of a valve annulus, delivering a plurality of coupled anchors from the anchor delivery device to secure the anchors to the annulus, and drawing the anchors together to circumferentially tighten the annulus. The device 1105 may include an elongate catheter having a housing at or near the distal end for releasably housing a plurality of coupled anchors 1115. The device may be positioned such that the housing abuts or is close to valve annular tissue 1102, such as at an intersection of the left ventricular wall and one or more mitral valve leaflets of the heart. Anchors 1115 may be drawn together to tighten the annulus by cinching a tether 1110 slidably coupled with the anchors. The device may include a steerable guide catheter for helping position the anchor delivery device for treating a valve annulus.

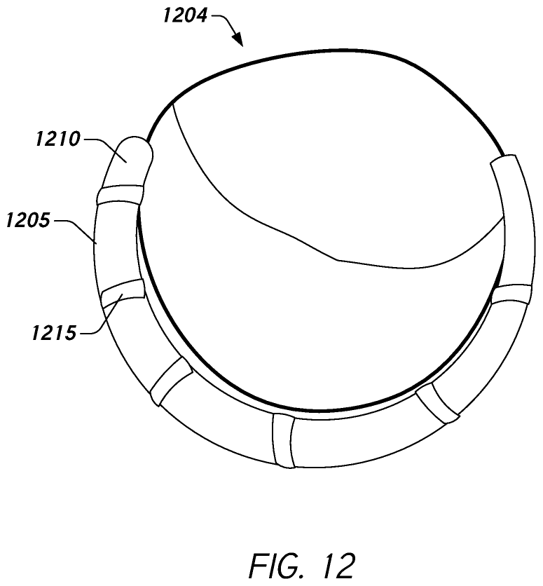

FIG. 12 illustrates using a high strength cord 1210 in an annuloplasty procedure that implants a band 1205 in the atrium at the annulus 1204, the high strength cord 1210 configured to cinch the band 1205 to reshape the annulus. The band 1205 includes a plurality of anchors 1215 to anchor the band 1205 in the atrium. In operation, the band 1205 functions similarly to the band 1105 described herein with reference to FIG. 11.

In some examples, a high strength cord can be used to close perforations in the ventricular or atrial septum.

### Examples of High Strength Cords

FIGS. 13A, 13B, and 13C illustrate examples of high strength cords 1310a, 1310b, 1310c using synthetic aramid polymer fibers (e.g., KEVLAR® aramid, TWARON® aramid, NOMEX® meta-aramid, etc.). FIG. 13A illustrates a high-strength cord 1310a comprising synthetic aramid polymer fibers 1311. The high-strength cord 1310a can be a bundle or assembly of individual filaments, sometimes referred to as a yarn. Each filament in the high-strength cord 1310a can be a synthetic aramid polymer filament.

FIG. 13B illustrates a high-strength cord 1310b comprising synthetic aramid polymer fibers 1311 with a coating 1312 configured to improve biostability. Because the disclosed high-strength cords are configured for use in the vascular system, a coating 1312 can be used to improve performance and/or to reduce degradation of the cord 1310b. Reduction in performance or degradation of an uncoated cord may occur due at least in part to the cord being exposed to the flow of blood. Thus, the coating 1312 can be configured to improve biostability and/or biocompatibility. Examples of such coatings include, for example and without limitation, hydrophilic coatings, hydrophobic coatings, or polymer sleeves or coatings (e.g., ultra-high-molecular-weight polyethylene (UHMWPE), polyethylene terephthalate (PET), polyether ether ketone (PEEK), polypropylene (PP), ePTFE, PTFE, or polystyrene (PS)).

Typically for cardiac repairs, ePTFE material is used for the sutures or cords. A benefit of using ePTFE material is that it promotes the formation of endothelial tissue due at least in part to its relatively high porosity. This is beneficial because endothelialization strengthens the ePTFE cords. Synthetic aramid polymer fibers, on the other hand, typically do not have the same level of porosity, potentially resulting in reduced endothelialization. This may not be an issue, though, because synthetic aramid polymer fibers are at least about 5 to 10 times stronger than ePTFE cords. Consequently, endothelialization may not significantly affect the durability or strength of cords made of synthetic aramid polymer fibers.

FIG. 13C illustrates a high-strength cord 1310c that is configured to mimic the porosity of ePTFE cords. This may be beneficial to promote endothelialization, where desirable. The high-strength cord 1310c includes synthetic aramid strands 1313 that are braided or twisted together. This can be done to promote tissue growth between the strands. In some examples, the high-strength cord 1310c can include additional structures 1314 (in addition to the synthetic aramid strands), e.g., a core structure 1314, to promote tissue growth between strands. The high-strength cord 1310c may also be coated with a coating to improve biostability, as described with reference to FIG. 13B. In some examples, the strands 1313 cover at least about 50% of the core structure 1314, at least about 60% of the core structure 1314, or at least about 70% of the core structure 1314. In some examples, the strands 1313 cover between about 50% and about 95% of the core structure 1314 or between about 60% and about 90% of the core structure 1314.

FIG. 14 illustrates an example of a high strength cord 1410 using a core 1411 of a high strength material and an external coating, jacket, or sheath 1415 of ePTFE material. The core 1411 can be made of a relatively high-strength material. The relatively high-strength material can include synthetic aramid polymer fibers, as described herein with respect to FIGS. 13A-13C. The relatively high-strength material may also include other high-strength materials. For example, the core 1411 can include composite materials such as carbon fiber or fiberglass. As another example, the core 1411 can include polymers such as UHMWPE, PET, PEEK, PP, or PS. As another example, the core 1411 can include metals such as stainless steel, titanium, or titanium alloys. As another example, the core 1411 can include ceramics such as silicon nitride or aluminum oxide.

The jacket 1415 can be made of ePTFE material. In some examples, the core 1411 is coated in ePTFE material thereby forming the jacket 1415. A variety of coating processes could be used such as braiding, wrapping, extruding, laminating, dipping, spraying, or spin coating the jacket material 1415 (e.g., ePTFE material) around the core material 1410. By surrounding the high-strength core 1411 with ePTFE material in the jacket 1415, the benefits of using the ePTFE material can be realized while also increasing the strength and durability of the cord 1410 (e.g., relative to a cord made primarily or exclusively of ePTFE material or polyester). For example, the ePTFE jacket 1415 can promote tissue growth or endothelialization, as described herein. This may improve or enhance the strength of the cord 1410. In addition, the ePTFE material can have a relatively low coefficient of friction thereby reducing wear. In some examples, the core 1411 can be inserted into the sheath 1415 that is formed without the core 1411, in other words, the core can be threaded through an inner lumen of the sheath 1415. In some examples, the jacket 1415 partially coats or encapsulates the core 1411 rather than fully coating or encapsulating the core 1411. In some examples, the jacket 1415 covers at least about 50% of the core 1411, at least about 60% of the core 1411, or at least about 70% of the core 1411. In some examples, the jacket 1415 covers between about 50% and about 95% of the core 1411 or between about 60% and about 90% of the core 1411.

In some examples, the core 1411 has a cross-section area that is at least about 80% of the total cross-section area of the cord 1410, at least about 85% of the total cross-section area of the cord 1410, or at least about 90% of the total cross-section area of the cord 1410. In certain examples, the sheath 1415 has a cross-section area that is less than or equal to about 20% of the total cross-section area of the cord 1410, less than or equal to about 15% of the total cross-section area of the cord 1410, or less than or equal to about 10% of the total cross-section area of the cord 1410.

FIG. 15 illustrates an example of a high strength cord 1510 using braided ePTFE strands 1513. The braided ePTFE strands form a tubular braid structure. This can be formed by crossing a number of strands of ePTFE material diagonally in such a way that each group of strands pass alternately over and under a group of strands laid up in the opposite direction. The strands 1513 can be PTFE or ePTFE monofilaments sutures. The strands 1513 can have a variety of cross-sectional shapes including, but not limited to, round, square, rectangular, etc. The strands 1513 can be intertwined using three or more parallel strands of ePTFE/PTFE sutures. The sutures 1513 can be interlaced in a variety of different patterns. These patterns influence the order of interlacing points in the braid structure and can affect the mechanical properties of the braid's structure. Areas in-between the braids can serve as a space for deposition and adhesion of endothelial cells. This added new layer of endothelialization, can be promising in improving durability. The high strength cord 1510 can exhibit increased mechanical strength with flexibility and softness and/or improved creep resistance under physiological cardiac tensions.

The use of braided ePTFE sutures can increase the mechanical strength, structural integrity, and durability of a cardiac repair by providing sufficient support to withstand the increase chordal tension associated with high intracardiac pressures. ePTFE is a material widely used in cardiac surgery and appreciated for its chemical inertness and biocompatibility.

FIG. 16 illustrates an example of a high strength cord 1610 using a core 1611 of a high strength material and an external sheath 1615 of ePTFE material, the sheath 1615 formed from ribbons of flattened ePTFE material 1616 wrapped around the core 1611. In some examples, the ePTFE can be flattened into a ribbon, wrapped around the core 1611, and heat fused to fuse the wrinkles together. In some examples, the core 1611 can be PET material and the sheath 1615 can be ePTFE. This combination of materials can provide desirable strength (e.g., due to the core material) and endothelialization (e.g., due to the ePTFE sheath).

In some examples, ePTFE material originally starts with a thickness of about 0.004″ that is then flattened to about 0.002″ using a cold working process, such as a jeweler's mill. The flattened ribbon 1616 is then wrapped around the core 1611 and fused to the core 1611 (e.g., using heat fusion). The overall diameter of the cord 1610 can be about 0.2 mm (about 0.01 inch) or less than or equal to about 0.5 mm (about 0.02 inch).

Example Methods for Repairing Cardiac Valves with High Strength Cords

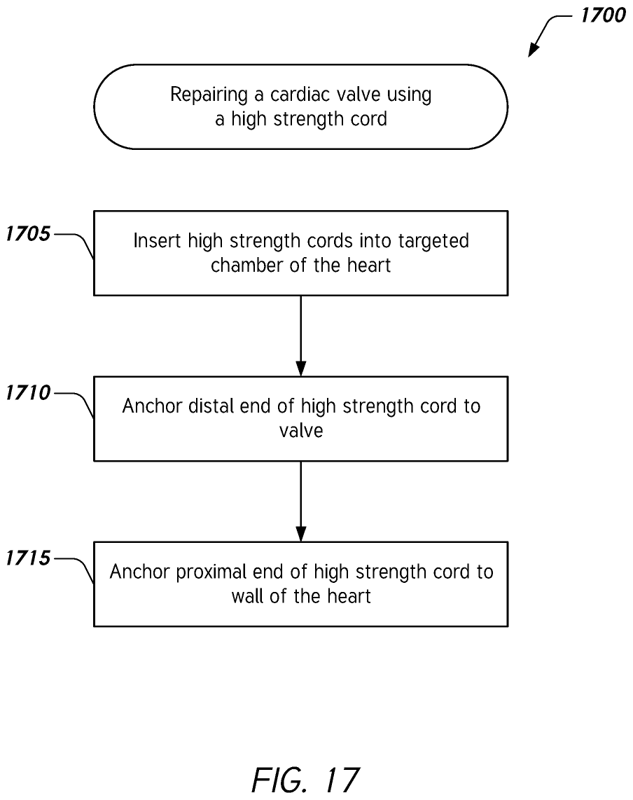

FIG. 17 illustrates a flowchart of an example method 1700 for repairing a cardiac valve (e.g., mitral valve, tricuspid valve, pulmonary valve, aortic valve) using any of the high-strength cords disclosed herein. In block 1705, a high strength cord is inserted into a targeted chamber (e.g., through a wall of the heart or using a transcatheter approach). For example, to repair the mitral valve, the high strength cord can be inserted through the ventricle. The high strength cord is any of the cords disclosed herein, including the cords described herein with respect to FIGS. 13-16.

In some examples, a delivery device can be used to deliver the high strength cord to the targeted chamber of the heart using a minimally invasive procedure. A piercing portion of the delivery device can be used to form an opening in the tissue, through which the distal end of the delivery device can be inserted. However, it is to be understood that any suitable procedure may be employed including an open-heart procedure, a less-invasive procedure, a non-invasive procedure, and/or a transcatheter approach.

In block 1710, a distal end of the high strength cord is anchored to the tissue of the targeted valve. When repairing a mitral valve, for example, the tissue of the targeted valve can include the posterior leaflet, the anterior leaflet, and/or the annulus of the valve. The distal end of the high strength cord can include a bulky knot as the anchor, examples of which are described herein with respect to FIG. 5. The distal end of the high strength cord can include any suitable anchor for securing the high strength cord to the tissue of the targeted valve, as described in greater detail herein.

The delivery device can be used to form or deliver a distal anchor to the distal side of the tissue of the targeted valve. The delivery device can be used in this manner to deliver two or more anchors to the distal side of the tissue. The anchors can be delivered to a single tissue (e.g., a posterior mitral valve leaflet), or one or more anchors can be delivered to a first tissue (e.g., a posterior mitral valve leaflet), and one or more other implants can be delivered to a second tissue (e.g., an anterior mitral valve leaflet, a mitral valve annulus, or any other suitable tissue) separate from the first tissue.

In block 1715, a proximal end of the high strength cord is anchored to the wall of the heart and/or a papillary muscle of the heart. To anchor the proximal end of the high strength cord, a pledget may be used.

The delivery device can then be withdrawn, and suture portions extending from the anchors can extend to a location (e.g., an outside surface of the heart or other suitable organ) remote from the tissue(s). The suture portions are the high strength cords that comprise synthetic aramid polymer fibers or a combination of a high strength material (composite material, polymer, ceramic, or metal) coated in ePTFE material, as disclosed herein. Where the term anchor is used herein, it is to be understood that an anchor refers to any suitable component or element that serves to anchor a suture to tissue such as, for example and without limitation, hooks, barbs, knots (e.g., bulky knots), and the like. In certain instances, the secured high strength cord can be suitably tensioned and/or pulled towards the access site, e.g., into the ventricle of the heart, resulting in a larger effective surface area of coaptation and improved coaptation between the leaflets.

The anchoring step is done to prevent or to reduce the likelihood that the sutures come loose. The high strength cords can be anchored to a tissue wall, such as an external wall of the heart. A pledget can be used as the anchor. For example, PTFE (TEFLON®, Dupont, Wilmington, Delaware) felt can be used as an anchor where the felt is attached to the tissue wall. In some examples, the anchor includes holes through which the high strength cord extends. Knots and/or locking sutures can be used to anchor the sutures.

In the methods disclosed herein, additional anchors and cords may be implanted. For example, to promote a larger surface of coaptation, anchors may be deployed in the body of the leaflets and/or at or near the annulus of the anterior and posterior leaflets, and the cords extending therefrom can be secured together and pulled to move the posterior annulus towards the anterior leaflet and/or the anterior annulus towards the posterior leaflet, thereby reducing the distance between the anterior annulus and the posterior annulus, e.g., the septal-lateral distance. Said another way, approximating the anterior annulus and the poster annulus in this manner can decrease the valve orifice, and thereby decreases, limits, or otherwise prevents undesirable regurgitation. One or more of the additional cords can be high strength cords and/or one or more of the additional cords can be of a different type (e.g., ePTFE sutures).

The method 1700 may be modified to reshape an internal organ rather than repairing a cardiac valve, an example of which is described herein with reference to FIG. 8. For example, the high strength cords can be anchored to enable the application of a force to move, shape, and/or remodel any part of an internal organ, such as the heart. The method 1700 may also be modified to include the implantation of an annuloplasty ring, an example of which is described herein with reference to FIG. 6. For example, the annuloplasty ring with a high-strength cord as a core material can be used to reduce the size of the annulus to improve performance of the valve (e.g., improve coaptation and/or to reduce regurgitation). Furthermore, the method 1700 may also be modified to manipulate papillary muscles to improve performance of the valve, generally referred to as sub-valvular techniques, an example of which is described herein with reference to FIG. 7. For example, a high strength cord can be secured to one or more papillary muscles and then used to relocate or otherwise manipulate the one or more papillary muscles with the purpose of improving valve performance. Furthermore, the method 1700 may also be modified to perform an annuloplasty procedure to reshape the annulus, examples of which are described herein with reference to FIGS. 9-12.

The above-described procedures can be performed manually, e.g., by a physician, or can alternatively be performed fully or in part with robotic or machine assistance. Further, although not specifically described herein, in various instances the heart may receive rapid pacing to reduce the relative motion of the edges of the valve leaflets during the procedures described herein (e.g., while an anchor, suture, and/or locking suture is being delivered and deployed).

### Additional Examples and Terminology

As used herein, the term aromatic polyamide and the term aramid refer to synthetic fibers where the chain molecules in the fibers are highly oriented along the fiber axis, resulting in a higher proportion of the chemical bonds contributing to fiber strength. Aramid fibers also include manufactured fibers in which the fiber-forming substance is a long-chain synthetic polyamide in which at least 85% of the amide linkages (—CO—NH—) are attached directly to two aromatic rings. Examples of aramids suitable for use in the disclosed cords include KEVLAR® aramid, TWARON® aramid, and NOMEX® meta-aramid.

While various examples have been described above, it should be understood that they have been presented by way of illustration only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or examples described above indicate certain components arranged in certain orientations or positions, the arrangement of components may be modified. While the examples have been particularly shown and described, it will be understood that various changes in form and details may be made. Any portion of the apparatus and/or methods described herein may be combined in any combination, except mutually exclusive combinations. The examples described herein can include various combinations and/or sub-combinations of the functions, components and/or features of the different examples described.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings provided herein can be applied to other methods and systems and are not limited to the methods and systems described above, and elements and acts of the various examples described above can be combined to provide further examples. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

What is claimed is:

1. A method for repairing a cardiac valve, the method comprising:
attaching a high strength cord to targeted tissue of a heart, the high strength cord including a distal anchor and a suture extending proximally from the distal anchor implant, the high strength cord having a tensile strength of at least 2000 MPa; and
anchoring a proximal end of the high strength cord to the heart.

2. The method of claim 1, wherein the high strength cord consists of synthetic aramid polymer fibers.

3. The method of claim 1, wherein the high strength cord comprises braided or twisted strands of a synthetic aramid polymer.

4. The method of claim 3, wherein the braided or twisted strands of the synthetic aramid polymer surround a core structure.

5. The method of claim 4, wherein the synthetic aramid strands cover at least 50% of the core structure.

6. The method of claim 1, wherein the high strength cord comprises a core portion of a high-strength material and a coating material coating the core portion, the coating material configured to improve biostability.

7. The method of claim 6, wherein the high-strength material comprises a high-strength polymer.

8. The method of claim 6, wherein the high-strength material comprises a metal.

9. The method of claim 6, wherein the high-strength material comprises a ceramic.

10. The method of claim 6, wherein the coating material comprises expanded polytetrafluoroethylene.

11. The method of claim 1, wherein the high strength cord comprises a core portion of a high strength material and a jacket portion surrounding the core portion.

12. The method of claim 11, wherein the high strength material comprises a high strength polymer.

13. The method of claim 11, wherein the high strength material comprises a metal.

14. The method of claim 11, wherein the high strength material comprises a ceramic.

15. The method of claim 11, wherein the jacket portion comprises expanded polytetrafluoroethylene.

16. The method of claim 15, wherein the jacket portion is formed from ribbons of flattened expanded polytetrafluoroethylene.

17. The method of claim 1, wherein anchoring the proximal end includes securing the proximal end to an external wall of the heart.

18. The method of claim 1, wherein anchoring the proximal end includes securing the proximal end to a papillary muscle of the heart.

19. The method of claim 1, wherein the targeted tissue includes a leaflet of a mitral valve.

20. The method of claim 1, wherein the distal anchor is a bulky knot formed using the high strength cord.
